(19) United States
(12) Patent Application Publication
(10) Pub. No.: US 2025/0199153 A1
(43) Pub. Date: Jun. 19, 2025

(54) METHODS AND SYSTEMS FOR TRACKING LIVING OBJECTS

(71) Applicant: Agency for Science, Technology and Research, Singapore (SG)

(72) Inventors: Yugang Ma, Singapore (SG); Bo Jin, Singapore (SG); Sumei Sun, Singapore (SG); Yonghong Zeng, Singapore (SG); Zhigang Zhong, Singapore (SG)

(21) Appl. No.: 18/851,605

(22) PCT Filed: Mar. 29, 2023
(86) PCT No.: PCT/SG2023/050208

§ 371 Date: Sep. 26, 2024

(30) Foreign Application Priority Data
Mar. 29, 2022 (SG) 10202203217Q

(51) Int. Cl. G01S 13/58; A61B 5/0205; A61B 5/024; A61B 5/0507; A61B 5/08; A61B 5/11; G01S 7/41; G01S 13/72; G01S 13/89

(52) U.S. Cl. CPC G01S 13/584; A61B 5/0205; A61B 5/0507; A61B 5/1117; G01S 13/726; G01S 13/89; A61B 5/02405; A61B 5/0816; G01S 7/415

(57) ABSTRACT

A method for tracking one or more living objects using at least one processor is provided. The method includes: receiving, via a plurality of receiving antennas, signals transmitted from a plurality of transmitting antennas; identifying a location of each of the one or more living objects with respect to a discrete time based on the signals received in a number of a sampling periods; determining a movement of each of the one or more living objects based on the identified location; determining one or more vital signs of each of the one or more living objects based on the movement of each of the one or more living objects; and tracking collectively the location and the one or more vital signs of each of the one or more living objects.

### CROSS-REFERENCE TO RELATED APPLICATION

This application claims the benefit of priority of Singapore Patent Application No. 10202203217Q filed on 29 Feb. 2022, the contents of which being hereby incorporated by reference in their entirety for all purposes.

### TECHNICAL FIELD

The present disclosure generally relates to methods and systems for tracking one or more living objects.

### BACKGROUND

In smart home and office environments, the presence, location, and behaviour of a human body are monitored according to its vital sign (VS). The VS itself can bring out a lot of intelligence functions and accordingly there is a large number of demands on it. For example, by detecting the human body's VS in the bedroom through the sensor embedded in the air-con, the sleeper's sleep quality can be recorded and presented to him or her, and the air-con temperature can also be intelligently adjusted according to the VSs detected. In bathrooms, fall (behaviour) detection with privacy protection is widely needed for accident alarm. In offices, by knowing the information of the human presence, and location according to VS, the office resource can be managed more efficiently.

Currently, VS detection needs contact sensors. It is not user friendly, because wearing sensors may not be convenient for all users in any environments, and sending detected VS to server needs wire connection or wireless transmitter. Another type of VS monitor based on video image needs video taking, this is strongly discouraged in bedrooms and prohibited in bathrooms.

A need therefore exists to provide a method and a system for tracking one or more living objects, that seek to overcome, or at least ameliorate, one or more deficiencies in conventional tracking methods and systems and more particularly, to improve efficiency (e.g., improving convenience) and/or effectiveness (e.g., enhancing tracking accuracy).

### SUMMARY

According to a first aspect of the present disclosure, there is provided a method for tracking one or more living objects using at least one processor is provided, the method including: receiving, via a plurality of receiving antennas, signals transmitted from a plurality of transmitting antennas; identifying a location of each of the one or more living objects with respect to a discrete time based on the signals received in a number of sampling periods; determining a movement of each of the one or more living objects based on the identified location; determining one or more vital signs of each of the one or more living objects based on the movement of each of the one or more living objects; and tracking collectively the location and the one or more vital signs of each of the one or more living objects.

According to a second aspect of the present disclosure, there is provided a method for tracking one or more living objects using at least one processor, the method including: receiving, via a plurality of receiving antennas, signals transmitted from a plurality of transmitting antennas; sampling the received signals to generate a sequence of received signal values for each pair of transmitting antenna and receiving antenna; generating frequency domain values by applying a Fourier Transform to the sequence of received signal values; detecting peaks in the frequency domain values with respect to a discrete time in a number of sampling periods; determining a phase perturbation of each detected peak to generate unwrapped phase angles; performing band pass filtering of the generated unwrapped phase angles for each peak; and determining a peak to correspond to a living object if a measure of the filtered unwrapped phase angles for the peak is above a predetermined threshold.

According to a third aspect of the present disclosure, there is provided a system for tracking living objects, the system including: at least one memory; and at least one processor communicatively coupled to the at least one memory and configured to perform the method as described herein.

According to a fourth aspect of the present disclosure, there is provided a non-transitory computer-readable storage medium, including instructions executable by at least one processor to perform the method as described herein.

### BRIEF DESCRIPTION OF THE DRAWINGS

Embodiments of the present disclosure will be better understood and readily apparent to one of ordinary skill in the art from the following written description, by way of example only, and in conjunction with the drawings, in which:

FIG. 1 depicts a schematic flow diagram of an example method for tracking one or more living objects, according to various embodiments of the present disclosure;

FIG. 2 depicts a schematic flow diagram of an example method for tracking one or more living objects, according to various embodiments of the present disclosure

FIG. 3 depicts a schematic block diagram of an example system for tracking one or more living objects, according to various embodiments of the present disclosure;

FIG. 4 depicts a schematic block diagram of an example system for tracking one or more living objects, according to various embodiments of the present disclosure;

FIG. 5 depicts a schematic block diagram of an exemplary computer system in which the system shown in FIG. 3, the first system shown in FIG. 4 or the second system shown in FIG. 4, according to various embodiments of the present disclosure, may be realized or implemented;

FIG. 6 depicts a schematic block diagram of an example method for tracking one or more living objects, according to various example embodiments of the present disclosure;

FIG. 7 depicts a schematic block diagram of an example method for tracking one or more living objects, according to various example embodiments of the present disclosure; and

FIG. 8 depicts a schematic block diagram of an example deep learning structure used in an example method for tracking one or more living objects, according to various example embodiments of the present disclosure.

### DETAILED DESCRIPTION

The following detailed description refers to the accompanying drawings that show, by way of illustration, specific details and aspects in which the disclosure may be practiced. One or more aspects are described in sufficient detail to enable those skilled in the art to practice the disclosure. Other aspects may be utilized and structural, logical, and/or electrical changes may be made without departing from the scope of the disclosure. The various aspects of the disclosure are not necessarily mutually exclusive, as some aspects can be combined with one or more other aspects to form new aspects or embodiments. Various aspects are described in connection with methods and various aspects are described in connection with devices. However, it may be understood that aspects described in connection with methods may similarly apply to the devices, and vice versa.

It should be understood that the singular terms “a”, “an”, and “the” include plural references unless context clearly indicates otherwise. Similarly, the word “or” is intended to include “and” unless the context clearly indicates otherwise.

It will be further understood that the terms “comprise” (and any form of comprise, such as “comprises” and “comprising”), “have” (and any form of have, such as “has” and “having”), “include” (and any form of include, such as “includes” and “including”), and “contain” (and any form of contain, such as “contains” and “containing”) are open-ended linking verbs. As a result, a method or device that “comprises,” “has,” “includes” or “contains” one or more steps or elements possesses those one or more steps or elements, but is not limited to possessing only those one or more steps or elements. Likewise, a step of a method or an element of a device that “comprises,” “has,” “includes” or “contains” one or more features possesses those one or more features, but is not limited to possessing only those one or more features. Furthermore, a device or structure that is configured in a certain way is configured in at least that way, but may also be configured in ways that are not listed.

As used herein, the phrase of the form of “at least one of A or B” may include A or B or both A and B. Correspondingly, the phrase of the form of “at least one of A or B or C”, or including further listed items, may include any and all combinations of one or more of the associated listed items.

The term “exemplary” may be used herein to mean “serving as an example, instance, or illustration”. Any aspect or design described herein as “exemplary” is not necessarily to be construed as preferred or advantageous over other aspects or designs.

The terms “at least one” and “one or more” may be understood to include a numerical quantity greater than or equal to one (e.g., one, two, three, four, [ . . . ], etc.). The term “a plurality” may be understood to include a numerical quantity greater than or equal to two (e.g., two, three, four, five, [ . . . ], etc.). The phrase “at least one of” with regard to a group of elements may be used herein to mean at least one element from the group consisting of the elements. For example, the phrase “at least one of” with regard to a group of elements may be used herein to mean a selection of: one of the listed elements, a plurality of one of the listed elements, a plurality of individual listed elements, or a plurality of a multiple of listed elements.

The words “plural” and “multiple” in the description and in the claims expressly refer to a quantity greater than one. Accordingly, any phrases explicitly invoking the aforementioned words (e.g., “a plurality of (objects)”, “multiple (objects)”) referring to a quantity of objects expressly refer to more than one of the said objects. The terms “group (of)”, “set (of)”, “collection (of)”, “series (of)”, “sequence (of)”, “grouping (of)”, etc., and the like in the description and in the claims, if any, refer to a quantity equal to or greater than one, i.e. one or more.

The term “first”, “second”, “third” detailed herein are used to distinguish one element from another similar element and may not necessarily denote order or relative importance, unless otherwise stated. For example, a first transaction data, a second transaction data may be used to distinguish two transactions based on two different foreign currency exchange.

The term “data” as used herein may be understood to include information in any suitable analog or digital form, e.g., provided as a file, a portion of a file, a set of files, a signal or stream, a portion of a signal or stream, a set of signals or streams, and the like. Further, the term “data” may also be used to mean a reference to information, e.g., in form of a pointer. The term “data”, however, is not limited to the aforementioned examples and may take various forms and represent any information as understood in the art. Any type of information, as described herein, may be handled for example via one or more processors in a suitable way, e.g. as data.

The terms “processor” or “controller” as, for example, used herein may be understood as any kind of entity that allows handling data. The data may be handled according to one or more specific functions executed by the processor or controller. Further, a processor or controller as used herein may be understood as any kind of circuit, e.g., any kind of analog or digital circuit. A processor or a controller may thus be or include an analog circuit, digital circuit, mixed-signal circuit, logic circuit, processor, microprocessor, Central Processing Unit (CPU), Graphics Processing Unit (GPU), Digital Signal Processor (DSP), Field Programmable Gate Array (FPGA), integrated circuit, Application Specific Integrated Circuit (ASIC), etc., or any combination thereof. Any other kind of implementation of the respective functions, which will be described below in further detail, may also be understood as a processor, controller, or logic circuit. It is understood that any two (or more) of the processors, controllers, or logic circuits detailed herein may be realized as a single entity with equivalent functionality or the like, and conversely that any single processor, controller, or logic circuit detailed herein may be realized as two (or more) separate entities with equivalent functionality or the like.

The term “memory” detailed herein may be understood to include any suitable type of memory or memory device, e.g., a hard disk drive (HDD), a solid-state drive (SSD), a flash memory, etc.

The term “module” detailed herein refers to, or forms part of, or includes an Application Specific Integrated Circuit (ASIC); an electronic circuit; a combinational logic circuit; a field programmable gate array (FPGA); a processor (shared, dedicated, or group) that executes code; other suitable hardware components that provide the described functionality; or a combination of some or all of the above, such as in a system-on-chip. The term module may include memory (shared, dedicated, or group) that stores code executed by the processor.

Differences between software and hardware implemented data handling may blur. A processor, controller, and/or circuit detailed herein may be implemented in software, hardware, and/or as a hybrid implementation including software and hardware.

The term “system” (e.g., a transaction facilitator system, a computing system, etc.) detailed herein may be understood as a set of interacting elements, wherein the elements can be, by way of example and not of limitation, one or more mechanical components, one or more electrical components, one or more instructions (e.g., encoded in storage media), and/or one or more processors, and the like.

Unless specifically stated otherwise, and as apparent from the following, it will be appreciated that throughout the present specification, description or discussions utilizing terms such as “performing”, “sampling”, “generating”, “determining”, “detecting” or the like, refer to the actions and processes of a computer system, or similar electronic device, that manipulates and transforms data represented as physical quantities within the computer system into other data similarly represented as physical quantities within the computer system or other information storage, transmission or display devices.

Some portions of the present disclosure are explicitly or implicitly presented in terms of algorithms and functional or symbolic representations of operations on data within a computer memory. These algorithmic descriptions and functional or symbolic representations are the means used by those skilled in the data processing arts to convey most effectively the substance of their work to others skilled in the art. An algorithm is here, and generally, conceived to be a self-consistent sequence of steps leading to a desired result. The steps are those requiring physical manipulations of physical quantities, such as electrical, magnetic or optical signals capable of being stored, transferred, combined, compared, and otherwise manipulated.

In this disclosure, a Multiple-Input and Multiple-Output (MIMO) vital sign (VS) radar system is proposed, which may identify multiple human bodies from all major targets in a room, and localize the respective human bodies as well as estimate VS including the breathing rate and heartbeat of every human body. The proposed system may provide comprehensive detections including target type identification, localization and VS estimations. Moreover, the unique joint localization and VS estimation solution may make use of the coherent information between the human body's moving speed and its VS, leading to reliable detections.

In this disclosure, an mmWave MIMO VS radar is disclosed. The proposed mmWave MIMO VS radar may address the technical needs and overcome the shortcoming of the existing solutions. It is compact enough to be integrated into ordinary consumer electronic appliances in resident homes and offices, e.g., air-con, refrigerator, TV, computer and so on. It is very convenient for large consumer electronics manufacturers to add values to their existing products by integrating with the proposed solution herein.

The following examples pertain to various aspects of the present disclosure.
Example 1 is a method for tracking one or more living objects using at least one processor, and including: receiving, via a plurality of receiving antennas, signals transmitted from a plurality of transmitting antennas; identifying a location of each of the one or more living objects with respect to a discrete time based on the signals received in a number of sampling periods; determining a movement of each of the one or more living objects based on the identified location; determining one or more vital signs of each of the one or more living objects based on the movement of each of the one or more living objects; and tracking collectively the location and the one or more vital signs of each of the one or more living objects.
In Example 2, the subject matter of Example 1 may optionally include that the one or more vital signs comprises a breathing rate and/or a heartbeat of the one or more living objects.
In Example 3, the subject matter of Example 2 may optionally include that the one or more vital signs further comprises a changing rate of the breathing rate and/or a changing rate of the heartbeat of the one or more living objects.
In Example 4, the subject matter of Example 1 may optionally include that the movement of each of the one or more living objects comprises information relating a speed of each of the one or more living objects.
In Example 5, the subject matter of Example 4 may optionally include that the information relating a speed of each of the one or more living objects comprises a Doppler speed of each of the one or more living objects and Direction of Arrival (DOA) estimation of each of the one or more living objects.
In Example 6, the subject matter of Example 2 may optionally include that determining one or more vital signs of each of the one or more living objects comprises: conducting a low pass filtering to obtain information relating to the breathing rate of the one or more living objects and/or conducting a high pass filtering to obtain information relating the heartbeat of the one or more living objects; and determining the breathing rate of the one or more living object by finding peaks in the information relating to the breathing rate of the one or more living objects during the sample period and/or determining the heartbeat of the one or more living objects by finding peaks in the information relating to the heartbeat of the one or more living objects during the sample period.
In Example 7, the subject matter of Example 6 may optionally include that the low pass filtering and the high pass filtering are conducted at a same frequency.
In Example 8, the subject matter of Example 6 may optionally include determining an intermediate parameter equal to the low pass filtered information relating to the breathing rate of the one or more living objects if the low pass filtered information is greater than a predetermined ratio of an average of the low pass filtered information during the sample period and determining the intermediate parameter to zero if the low pass filtered information is less than or equal to predetermined ratio of the average of the low pass filtered information; and/or determining an intermediate parameter equal to the high pass filtered information relating to the heartbeat of the one or more living objects if the high pass filtered information is greater than a predetermined ratio of an average of the high pass filtered information during the sample period and determining the intermediate parameter to zero if the high pass filtered information is less than or equal to predetermined ratio of the average of the high pass filtered information.
In Example 9, the subject matter of Example 1 may optionally include detecting a fall by using the convolutional neural network (CNN).
In Example 10, the subject matter of Example 1 may optionally include that identifying a location of each of the one or more living objects based on the signals received in a number of sampling periods comprises: sampling the received signals to generate a sequence of received signal values for each pair of transmitting antenna and receiving antenna; generating frequency domain values by applying a Fourier Transform to the sequence of received signal values; generating a radar imaging result by applying a spatial spectrum recovering algorithm to the frequency domain values to generate a first radar imaging result; detecting peaks in the radar imaging result; determining a phase perturbation of each detected peak to generate unwrapped phase angles; performing band pass filtering of the generated unwrapped phase angles for each peak; and determining a peak to correspond to a living object if a measure of the filtered unwrapped phase angles for the peak is above a predetermined threshold.
In Example 11, the subject matter of Example 1 may optionally include displaying the location of each of the one or more living objects in a target location map; and indicating a location for each of one or more non-living object in the target location map.
In Example 12, the subject matter of Example 1 may optionally include displaying waveforms of the vital signs of the one or more living objects in accordance with estimated rates thereof.
In Example 13, the subject matter of Example 1 may optionally include, for every living object of the one or more living objects; simultaneously identifying the location with respect to the discrete time based on the signals received in a number of sampling periods; simultaneously determining the movement based on the identified location; simultaneously determining the one or more vital signs based on the movement; and simultaneously tracking collectively the location and the one or more vital signs using Kalman filtering.
Example 14 is a method for tracking one or more living objects using at least one processor, the method comprising: receiving, via a plurality of receiving antennas, signals transmitted from a plurality of transmitting antennas; sampling the received signals to generate a sequence of received signal values for each pair of transmitting antenna and receiving antenna; generating frequency domain values by applying a Fourier Transform to the sequence of received signal values; detecting peaks in the frequency domain values with respect to a discrete time in a number of sampling periods; determining a phase perturbation of each detected peak to generate unwrapped phase angles; performing band pass filtering of the generated unwrapped phase angles for each peak; and determining a peak to correspond to a living object if a measure of the filtered unwrapped phase angles for the peak is above a predetermined threshold.
In Example 15, the subject matter of Example 14 may optionally include, prior to detecting peaks in the frequency domain values, generating a radar imaging result by applying a spatial spectrum recovering algorithm to the frequency domain values to generate a first radar imaging result; wherein the spatial spectrum recovering algorithm gives a preliminary radar imaging result and generating the radar imaging result further comprises applying a constant false alarm rate (CFAR) algorithm to the preliminary radar imaging result.
In Example 16, the subject matter of Example 14 may optionally include determining Doppler domain values of the frequency domain values.
In Example 17, the subject matter of Example 14 may optionally include conducting a low pass filtering with a third frequency on the filtered unwrapped phase angles to generate raw breath rates; modulating a breath rate data equal to the raw breath rate if the raw breath rate is greater than a predetermined ratio of an average of the raw breath rates during a sampling period and determining a breath rate data to zero if the raw breath rate is less than or equal to predetermined ratio of the average of the raw breath rates during the sampling period; and determining a breath rate of a living object by finding peaks in the breath rate data during the sampling period and sizing the number of the peaks of the breath rate data.
In Example 18, the subject matter of Example 14 may optionally include conducting a high pass filtering with a fourth frequency on the filtered unwrapped phase angles to generate raw heartbeats; modulating a heartbeat data equal to the raw heartbeat if the raw heartbeat is greater than a predetermined ratio of an average of the raw heartbeats during a sampling period and modulating a heartbeat data to zero if the raw heartbeat is less than or equal to predetermined ratio of the average of the raw heartbeats during the sampling period; and determining a heartbeat of a living object by finding peaks in the heartbeat data during the sampling period and sizing the number of the peaks of the heartbeat data.
In Example 19, the subject matter of Example 14 may optionally include forming zoom-in range-time map and zoom-in range-Doppler map; and processing the maps by deep learning structure.
Example 20 is a system for tracking living objects, the system comprising: at least one memory; and at least one processor communicatively coupled to the at least one memory and configured to perform the method according to any one of Examples 1 to 13 or the method according to any one of Examples 14 to 19.
Example 21 is a non-transitory computer-readable storage medium, comprising instructions executable by at least one processor to perform the method according to any one of Examples 1 to 13 or the method according to any one of Examples 14 to 19.

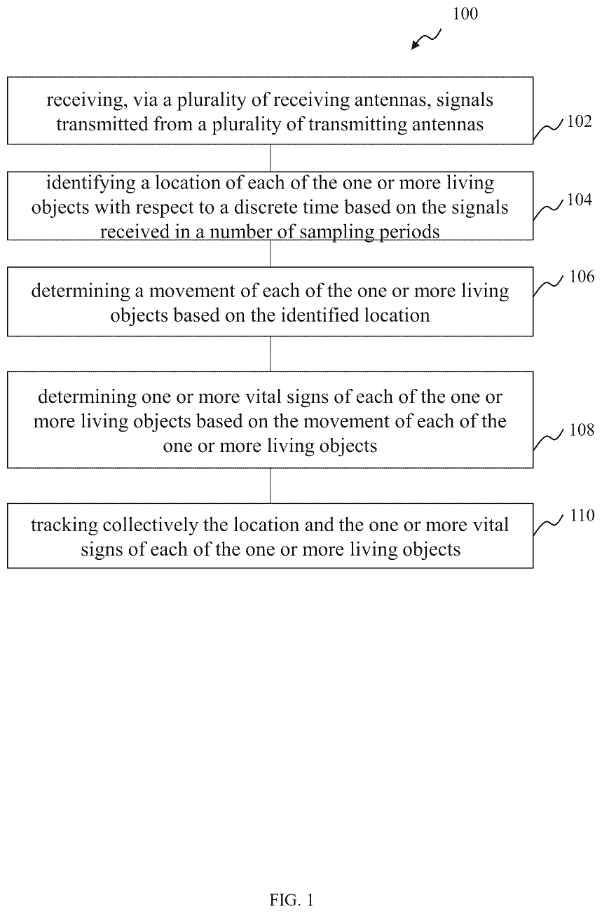

FIG. 1 depicts a schematic flow diagram of a method 100 for tracking one or more living objects according to various embodiments of the present disclosure. The method 100 includes: receiving (at step 102), via a plurality of receiving antennas, signals transmitted from a plurality of transmitting antennas; identifying (at step 104) a location of each of the one or more living objects with respect to a discrete time based on the signals received in a number of sampling periods; determining (at step 106) a movement of each of the one or more living objects based on the identified location; determining (at step 108) one or more vital signs of each of the one or more living objects based on the movement of each of the one or more living objects; and tracking (at step 110) collectively the location and the one or more vital signs of each of the one or more living objects using filtering.

According to various non-limiting embodiments, the plurality of receiving antennas and the plurality of transmitting antennas may include radar antennas receiving and transmitting radar waves (e.g. microwaves) and accordingly the signals may be radar signals (e.g. radar waves). The plurality of receiving antennas may function as the plurality of transmitting antennas, that is, the plurality of receiving antennas may be the same as the plurality of transmitting antennas. The signals transmitted by the plurality of transmitting antennas may reach objects which in turn reflect or scatter the signals, and the reflected or scattered signals may be received by the plurality of receiving signals. The receiving signals may contain information about the objects including, but not limited to, the range, velocity of the objects.

According to various non-limiting embodiments, the plurality of receiving antennas and the plurality of transmitting antennas may follow multiple-input and multiple-output (MIMO) method. That may mean multiplying the capacity of a radio link using the plurality of transmitting and receiving antennas to exploit multipath propagation.

According to various non-limiting embodiments, an analog-to-digital (ADC) converter may be utilized to convert continuous-time and continuous-amplitude analog signals from the antennas to discrete-time and discrete-amplitude digital signals. The digital signals in a number of sampling periods may be processed by the method as described herein to obtain the location of each of the one or more living objects with respect to a discrete time from which the number of sampling periods starts. In other words, the location of each of the one or more living objects with respect to the discrete time may be obtained based on the digital signals received in the number of sampling periods.

According to various non-limiting embodiments, a movement of each of the one or more living objects may be determined based on the identified location (e.g. with respect to the discrete time based on the signals received in the number of sampling periods). In some embodiments, the movement may include information relating a speed of the living object, for example, velocity of the living object detected by the plurality of receiving antennas. In some embodiments, the movement may include information relating to a Doppler speed determined by applying Fast-Fourier Transform (FFT) to the identified location in Doppler domain and Direction of Arrival (DOA) estimation of each of the one or more living objects. Doppler FFT may be for target moving speed and represent the speed relative to the radar instead of speeds along X and Y axes.

According to various non-limiting embodiments, one or more vital signs of each of the one or more living objects may be determined based on the movement of each of the one or more living objects. The one or more vital signs of living objects may include breathing rate, heartbeat, pulse rate, respiration rate and the like, or a changing rate of the above. The signals may be further processed (e.g. smoothed, modulated) by removing noises and interferences. The signals may be also processed by band pass filtering (e.g. high pass filtering and/or low pass filtering) so as to separate information relating to each of the one or more vital signs.

In various embodiments, determining one or more vital signs of each of the one or more living objects may include: conducting a low pass filtering to obtain information relating to the breathing rate of the one or more living objects, and determining the breathing rate of the one or more living object by finding peaks in the information relating to the breathing rate of the one or more living objects during the sample period. Further, determining one or more vital signs of each of the one or more living objects may include determining an intermediate parameter equal to the low pass filtered information relating to the breathing rate of the one or more living objects if the low pass filtered information is greater than a predetermined ratio of an average of the low pass filtered information during the sample period and determining the intermediate parameter to zero if the low pass filtered information is less than or equal to predetermined ratio of the average of the low pass filtered information.

In various embodiments, determining one or more vital signs of each of the one or more living objects may include: conducting a high pass filtering to obtain information relating the heartbeat of the one or more living objects; and determining the heartbeat of the one or more living objects by finding peaks in the information relating to the heartbeat of the one or more living objects during the sample period. Further, determining one or more vital signs of each of the one or more living objects may include: determining an intermediate parameter equal to the high pass filtered information relating to the heartbeat of the one or more living objects if the high pass filtered information is greater than a predetermined ratio of an average of the high pass filtered information during the sample period and determining the intermediate parameter to zero if the high pass filtered information is less than or equal to predetermined ratio of the average of the high pass filtered information.

In various embodiments, the low pass filtering and the high pass filtering may be conducted at a same frequency. For example, at a frequency of 0.5 Hz.

According to various non-limiting embodiments, the location and the one or more vital signs of each of the one or more living objects may be tracked collectively or jointly. In some embodiments, the movement and/or changing rate of the one or more vital signs of each of the one or more living objects may be tracked collectively or jointly. The tracking may include estimating the location and the one or more vital signs of each of the one or more living objects with variance or uncertainty of the estimate. In some embodiments, the location and the one or more vital signs of each of the one or more living objects may be tracked collectively or jointly using Kalman filtering. Specifically, the Kalman filtering may track the location, the breathing rates and heartbeats, the movement by tracking the speed of the object movement, and changing rates of the breathing rates and heartbeats of the one or more living objects with respect to subsequent discrete times. That is, Kalman filtering may update the location, the breathing rates and heartbeats, the movement by tracking the speed of the object movement, and changing rates of the breathing rates and heartbeats of the one or more living objects with respect to a succeeding discrete time based on signals received in a number of sampling periods starting from the succeeding discrete time.

According to various non-limiting embodiments, the method 100 may include detecting a fall by using the convolutional neural network (CNN). The method 100 may include tracking a sudden change of the speed of the object movement. The sudden change may mean the change of the speed above a threshold. The threshold may be predetermined or machine-learnt from the received signals.

In various embodiments, identifying a location of each of the one or more living objects with respect to a discrete time based on the signals received in a number of sampling periods may include: sampling the received signals to generate a sequence of received signal values for each pair of transmitting antenna and receiving antenna; generating frequency domain values by applying a Fourier Transform to the sequence of received signal values; generating a radar imaging result by applying a spatial spectrum recovering algorithm to the frequency domain values to generate a first radar imaging result; detecting peaks in the radar imaging result; determining a phase perturbation of each detected peak to generate unwrapped phase angles; performing band pass filtering of the generated unwrapped phase angles for each peak; and determining a peak to correspond to a living object if a measure of the filtered unwrapped phase angles for the peak is above a predetermined threshold.

In various embodiments, the method 100 may further include displaying the location of each of the one or more living objects in a target location map; and indicating a location for each of one or more non-living object in the target location map. The target location map may include a radar imaging map.

In various embodiments, the method 100 may further include displaying waveforms of the vital signs of the one or more living objects in accordance with estimated rates thereof. The waveforms of the vital signs of the one or more living objects may be formed by beamforming based on the received signals. The estimated rates of the one or more living objects may include the jointly or collectively tracked location and one or more vital signs of each of the one or more living objects.

In various embodiments, the method may include, for every living object of the one or more living objects: simultaneously identifying the location with respect to a discrete time based on the signals received in a number of sampling periods; simultaneously determining the movement based on the identified location; simultaneously determining the one or more vital signs based on the movement; and simultaneously tracking collectively the location and the one or more vital signs using Kalman filtering. By “simultaneously”, it is intended to include tracking each of the one or more objects at the same time, for example, the received signals at each discrete time (e.g. the signals received in the number of sampling periods starting from each discrete time) include information for each of the one or more objects; it is also intended to include tracking each of the one or more objects at consecutive discrete times while a time interval between two immediate consecutive discrete times is set to below a threshold.

Accordingly, the method 100 for tracking one or more living objects according to various embodiments of the present disclosure advantageously enables jointly tracking location and vital signs of the one or more living objects in a more efficient and effective manner. These advantages or technical effects, and/or other advantages or technical effects, will become more apparent to a person skilled in the art as the methods and systems for tracking one or more living objects, are described in more details according to various embodiments and example embodiments of the present disclosure.

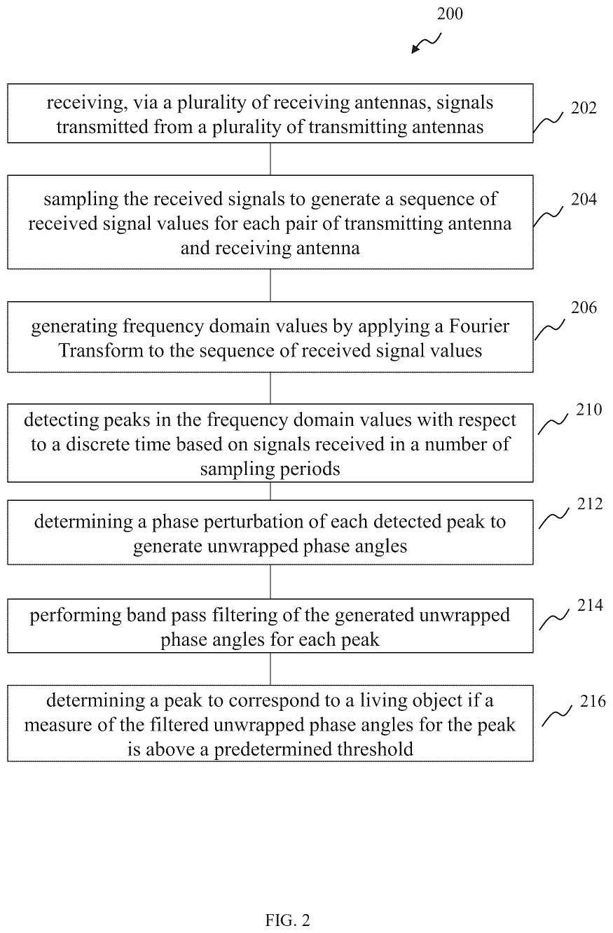

FIG. 2 depicts a schematic flow diagram of a method 200 for tracking one or more living objects, according to various embodiments of the present disclosure. The method 200 comprises: receiving (at step 202), via a plurality of receiving antennas, signals transmitted from a plurality of transmitting antennas; sampling (at step 204) the received signals to generate a sequence of received signal values for each pair of transmitting antenna and receiving antenna; generating (at step 206) frequency domain values by applying a Fourier Transform to the sequence of received signal values; detecting (at step 210) peaks in the frequency domain value with respect to the discrete time in the number of sampling periods; determining (at step 212) a phase perturbation of each detected peak to generate unwrapped phase angles; performing (at step 214) band pass filtering of the generated unwrapped phase angles for each peak; and determining (at step 216) a peak to correspond to a living object if a measure of the filtered unwrapped phase angles for the peak is above a predetermined threshold.

Features that are described in the context of the method 100 may correspondingly be applicable to the same or similar features in the method 200. Furthermore, additions and/or combinations and/or alternatives as described for a feature in the context of the method 100 may correspondingly be applicable to the same or similar feature in the method 200.

According to various non-limiting embodiments, the plurality of transmitting antennas may be the same as the plurality of receiving antennas. Accordingly, the pair of transmitting antenna and receiving antenna may the same one antenna. That is, a signal transmitted by a respective transmitting antenna of the plurality of transmitting antennas is received by the respective transmitting antenna when it functions as a corresponding receiving antenna of the plurality of receiving antennas.

According to various non-limiting embodiments, the received signals may be sampled to generate a sequence of received signal values for each pair of transmitting antenna and receiving antenna.

In various embodiments, the sequence of received signal values may be reconstructed to an antenna array signal following MIMO principle, for example, multiple receiving antennas and multiple transmitting antennas. That may mean that the received signals (e.g. the sequence of received signal values) may form a matrix with the number of matrix rows being the number of receiving antennas and the number of matrix columns being the number of transmitting antennas. That may also mean performing vectorization on the matrix of received signals to obtain a column vector as the reconstructed antenna array signal. The column vector may be obtained with respect to the sample of the analog-to-digital conversion (ADC) output.

According to various non-limiting embodiments, generating frequency domain values by applying a Fourier Transform to the sequence of received signal values may include generating frequency domain values by applying a Fourier Transform to the reconstructed antenna array signal. The Fourier Transform may include Fast Fourier Transform (FFT). A range FFT length may be preset. Applying a Fourier Transform to the sequence of received signal values may include applying row-wise 1-dimension (1D) Fourier Transform to the sequence of received signal values. That is, applying the Fourier Transform to a column vector for each channel of antenna array (e.g. each row of the matrix of received signals). The frequency domain values may be generated with respect to blocks, each of which taking a preset number of sequential samples.

According to various non-limiting embodiments, prior to detecting peaks in the frequency domain values, the method 200 may further include: generating a radar imaging result by applying a spatial spectrum recovering algorithm to the frequency domain values to generate a first radar imaging result. A radar imaging result may be generated by applying a spatial spectrum recovering algorithm to the frequency domain values to generate a first radar imaging result. The radar imaging result may be generated with respect to a discrete time based on signals received in a number of sampling periods. The spatial spectrum recovering algorithm may include process obtaining spectral information and/or spatial information from relevant data, for example, Capon method, Steering vector beamforming.

In various embodiments, a steering vector may be determined to represent a set of phase-delays for an incoming wave at each receiving antenna. The steering vector may be fixed for the plurality of receiving antennas. The steering vector may correspond to transmission frequency of the plurality of transmitting antennas.

In various embodiments, a frequency domain value matrix may be formed containing the elements of the frequency domain values with respect to the sampling period.

In various embodiments, a spatial covariance matrix may be determined with respect to the discrete time based on the signals received in a number of sampling periods. The spatial covariance matrix may be determined as a function of the frequency domain value matrix and a conjugated transpose of the frequency domain value matrix.

In various embodiments, the first radar imaging result (i.e., the first radar image mapped from the first radar imaging result into a 2D image) may be generated based on the steering vector, the frequency domain value matrix and the spatial covariance matrix. The first radar imaging result (i.e., the first radar image) may be further generated in accordance with an angle scanning step size (e.g., 1 degree) of the antennas. The first radar image may be generated with respect to a discrete time and updated every preset number of sequential samples from all antenna array channels. The size of the first radar image may correspond to the number of the antennas as well as the number of sequential samples, e.g., a product of the number of the antennas and the number of sequential samples.

In various embodiments, the first radar imaging result may be further improved to generate the radar imaging result, for example, by constant false alarm rate (CFAR) operation. Improvement on the first radar imaging result may include removing unwanted background noise signals by threshold values of the first radar imaging result within a present block size. Similarly, the radar imaging result (i.e. the radar image) may be generated with respect to a discrete time and updated every preset number of sequential samples from all antenna array channels. The size of the radar image may correspond to the number of the antennas as well as the number of sequential samples, e.g., a product of the number of the antennas and the number of sequential samples.

According to various non-limiting embodiments, peak locations in the radar image may be identified as detection objects (e.g. target locations). The peak locations may include locations for living objects and non-living objects.

According to various non-limiting embodiments, information relating to peak locations may be further analyzed to determine if the peak corresponds to a living object. A phase perturbation of each detected peak may be determined to generate unwrapped phase angles. That may mean analyzing the phase perturbation (e.g. phase delay, phase shifting) of the received signals during the sampling period.

According to various non-limiting embodiments, band pass filtering with one or more frequencies may be performed for the generated unwrapped phase angles for each peak. The band pass filtering may be performed with two cut-off frequencies, e.g., a lower bound (e.g., lowest) frequency and a higher bound (e.g., highest) frequency, so as to filter out undesirable signals, e.g., from non-living objects as interference. The interference may include, but not limited to, the hardware circuit caused low frequency drift as well as non-living moving object caused high frequency varying. For example, the band pass filtering may be performed with a first frequency of 0.1 Hz and a second frequency of 4 Hz. That may mean any signals with varying frequency lower than 0.1 Hz or higher than 4 Hz that are unlikely from living objects (e.g., human body) are filtered out by the band pass filtering. In other words, signals relating to vital signs of living objects (e.g., human body) may be retained after the band pass filtering with the two cut-off frequencies, e.g., a lower bound (e.g., lowest) frequency and a higher bound (e.g., highest) frequency.

According to various non-limiting embodiments, a peak may be determined to correspond to a living object if a measure of the filtered unwrapped phase angles for the peak is above a predetermined threshold. The measure of the filtered unwrapped phase angles may include taking the norm of the quantity of the filtered unwrapped phase angles.

In various embodiments, the method 200 may further include determining Doppler domain values of the frequency domain values. The Doppler domain values may include Doppler speeds.

In various embodiments, the method 200 may further include conducting a low pass filtering with a third frequency on the filtered unwrapped phase angles to generate raw breath rates; modulating a breath rate data equal to the raw breath rate if the raw breath rate is greater than a predetermined ratio of an average of the raw breath rates during a sampling period and determining a breath rate data to zero if the raw breath rate is less than or equal to predetermined ratio of the average of the raw breath rates during the sampling period; and determining a breath rate of a living object by finding peaks in the breath rate data during the sampling period and sizing the number of the peaks of the breath rate data.

In various embodiments, the method 200 may further include conducting a high pass filtering with a fourth frequency on the filtered unwrapped phase angles to generate raw heartbeats; modulating a heartbeat data equal to the raw heartbeat if the raw heartbeat is greater than a predetermined ratio of an average of the raw heartbeats during a sampling period and modulating a heartbeat data to zero if the raw heartbeat is less than or equal to predetermined ratio of the average of the raw heartbeats during the sampling period; and determining a heartbeat of a living object by finding peaks in the heartbeat data during the sampling period and sizing the number of the peaks of the heartbeat data.

In various embodiments, the third frequency may be the same as the fourth frequency. In various embodiments, the third frequency may be the less than the fourth frequency, for example, by a difference of 0.05 Hz. Accordingly, the application of low pass filtering to obtain the breath rates and high pass filtering to obtain the heartbeats may overcome the overlapping pass-band issue of the breadth rates and heartbeats.

While the methods 100, 200 described above is illustrated and described as a series of steps or events, it will be appreciated that any ordering of such steps or events are not to be interpreted in a limiting sense. For example, some steps may occur in different orders and/or concurrently with other steps or events apart from those illustrated and/or described herein. In addition, not all illustrated steps may be required to implement one or more aspects or embodiments described herein. Also, one or more of the steps depicted herein may be carried out in one or more separate acts and/or phases.

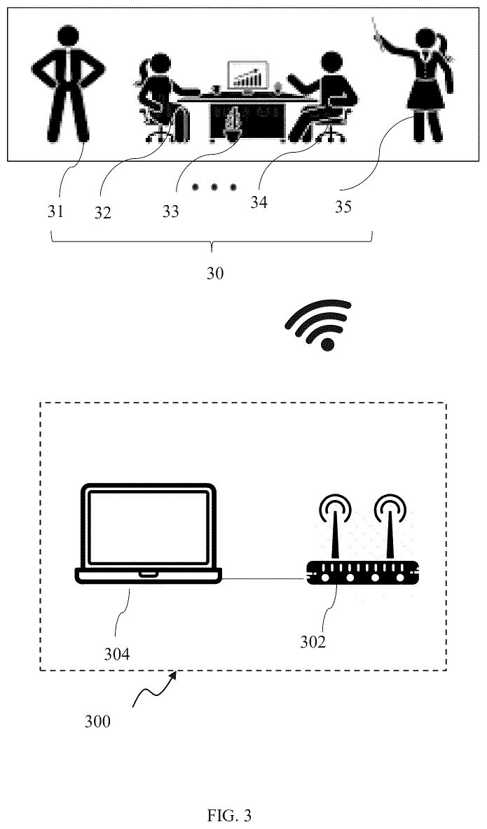

FIG. 3 depicts a schematic block diagram of a system 300 for tracking one or more living objects according to various embodiments of the present disclosure, corresponding to the above-mentioned method 100 for tracking one or more living objects as described hereinbefore according with reference to FIG. 1 according to various embodiments of the present disclosure. The system 300 comprises: at least one receiver 302; and at least one processor 304 communicatively coupled to the at least one receiver 302 and configured to perform the method 100 for tracking one or more living objects as described hereinbefore according to various embodiments of the present disclosure. Accordingly, the at least one receiver 302 is configured to: receiving (at step 102), via the receiver 302 (e.g. a plurality of receiving antennas), signals transmitted from a plurality of transmitting antennas (e.g. the receiver 302). The signals may include information relating to all the objects 30 including living objects 31, 32, 34, 35 and non-living objects, for example 34, in an area as shown in FIG. 3. The living object 31 and/or 35 may move around in the area during a sampling period. It should be appreciated that FIG. 3 only show an exemplary area where the system 300 is implemented, any other area may be applicable, for example, a ward room in a hospital, a bedroom, a bathroom or the like where vital signs of living objects are desirable to be tracked.

Accordingly, the at least one processor 304 is configured to: identifying (at step 104) a location of each of the one or more living objects (e.g. living objects 31, 32, 34, 35) with a discrete time based on the signals received in a number of sampling periods: determining (at step 106) a movement of each of the one or more living objects based on the identified location; determining (at step 108) one or more vital signs of each of the one or more living objects based on the movement of each of the one or more living objects; and tracking (at step 110) collectively the location and the one or more vital signs of each of the one or more living objects using filtering.

It will be appreciated by a person skilled in the art that the at least one processor 304 may be configured to perform various functions or operations through set(s) of instructions (e.g., software modules) executable by the at least one processor 304 to perform various functions or operations. Accordingly, the processor 304 of the system 300 may comprise: a data processing module (not shown) configured to process the signals received by the receiver 302 so as to track the one or more living objects.

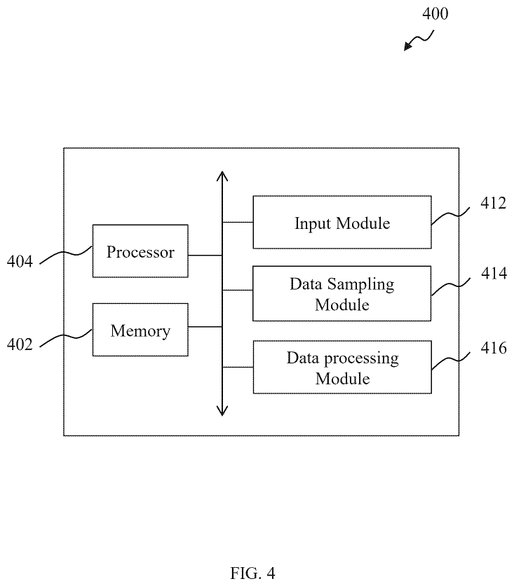

FIG. 4 depicts a schematic block diagram of a system 400 for tracking one or more living objects according to various embodiments of the present disclosure, corresponding to the above-mentioned method 200 for tracking one or more living objects as described hereinbefore according with reference to FIG. 2 according to various embodiments of the present disclosure. The system 400 comprises: at least one memory 402; and at least one processor 404 communicatively coupled to the at least one memory 402 and configured to perform the method 200 for tracking one or more living objects as described hereinbefore according to various embodiments of the present disclosure. Accordingly, the at least one processor 404 is configured to; receiving (at step 202), via a plurality of receiving antennas, signals transmitted from a plurality of transmitting antennas; sampling (at step 204) the received signals to generate a sequence of received signal values for each pair of transmitting antenna and receiving antenna; generating (at step 206) frequency domain values by applying a Fourier Transform to the sequence of received signal values; detecting (at step 210) peaks in the frequency domain values with respect to the discrete time in the number of sampling periods; determining (at step 212) a phase perturbation of each detected peak to generate unwrapped phase angles; performing (at step 214) band pass filtering of the generated unwrapped phase angles for each peak; and determining (at step 216) a peak to correspond to a living object if a measure of the filtered unwrapped phase angles for the peak is above a predetermined threshold.

It will be appreciated by a person skilled in the art that the at least one processor 404 may be configured to perform various functions or operations through set(s) of instructions (e.g., software modules) executable by the at least one processor 404 to perform various functions or operations. Accordingly, as shown in FIG. 4, the system 400 may comprise: an input module (or an input circuit) 412 configured to perform the above-mentioned receiving (at step 202), via a plurality of receiving antennas, signals transmitted from a plurality of transmitting antennas; a data sampling module (or a data sampling circuit) 414 configured to perform the above-mentioned sampling (at step 204) the received signals to generate a sequence of received signal values for each pair of transmitting antenna and receiving antenna; and a data processing module (or a data processing circuit) 416 configured to perform the above-mentioned generating (at step 206) frequency domain values by applying a Fourier Transform to the sequence of received signal values; detecting (at step 210) peaks in the frequency domain values with respect to a discrete time in a number of sampling periods; determining (at step 212) a phase perturbation of each detected peak to generate unwrapped phase angles; performing (at step 214) band pass filtering of the generated unwrapped phase angles for each peak; and determining (at step 216) a peak to correspond to a living object if a measure of the filtered unwrapped phase angles for the peak is above a predetermined threshold.

It will be appreciated by a person skilled in the art that various modules of a system are not necessarily separate modules, and two or more modules may be realized by or implemented as one functional module (e.g., a circuit or a software program) as desired or as appropriate without deviating from the scope of the present disclosure. For example, two or more modules of the system 400 for tracking one or more living objects (e.g., the input module 412, the data sampling module 414 and the data processing module 416) may be realized (e.g., compiled together) as one executable software program (e.g., software application or simply referred to as an “app”), which for example may be stored in the at least one memory 402 and executable by the at least one processor 404 to perform various functions/operations as described herein according to various embodiments of the present disclosure.

It will be appreciated by a person skilled in the art that a system may include further modules, for example, the system 400 may include a display module (not shown) configured to displaying the location of each of the one or more living objects in a target location map and/or displaying waveforms of the vital signs of the one or more living objects in accordance with estimated rates thereof according to the steps as described therein.

In various embodiments, the system 300 for tracking one or more living objects may correspond to the method 100 for tracking one or more living objects as described hereinbefore with reference to FIG. 1 according to various embodiments, therefore, various functions or operations configured to be performed by the least one processor 304 may correspond to various steps or operations of the method 100 for tracking one or more living objects as described hereinbefore according to various embodiments, and thus need not be repeated with respect to the system 300 for tracking one or more living objects for clarity and conciseness. In other words, various embodiments described herein in context of the methods are analogously valid for the corresponding systems, and vice versa.

For example, in various embodiments, the system may further include at least one memory (not shown) stored therein the data processing module, which correspond to one or more steps (or operation(s) or function(s)) of the method 100 for tracking one or more living objects as described herein according to various embodiments, which are executable by the at least one processor 304 to perform the corresponding function(s) or operation(s) as described herein.

Similarly, in various embodiments, the system 400 for tracking one or more living objects corresponds to the method 200 for tracking one or more living objects as described hereinbefore with reference to FIG. 2 according to various embodiments, therefore, various functions or operations configured to be performed by the least one processor 404 may correspond to various steps or operations of the method 200 for tracking one or more living objects as described hereinbefore according to various embodiments, and thus need not be repeated with respect to the system 400 for tracking one or more living objects for clarity and conciseness.

For example, in various embodiments, the at least one memory 402 of the system 400 may have stored therein the input module 412, the data sampling module 414, and/or the data processing module 416, which correspond to one or more steps (or operation(s) or function(s)) of the method 200 for tracking one or more living objects as described herein according to various embodiments, which are executable by the at least one processor 404 to perform the corresponding function(s) or operation(s) as described herein.

A computing system, a controller, a microcontroller or any other system providing a processing capability may be provided according to various embodiments in the present disclosure. Such a system may be taken to include one or more processors and one or more computer-readable storage mediums. For example, the system 400 for tracking one or more living objects described hereinbefore may include at least one processor (or controller) 404 and at least one computer-readable storage medium (or memory) 402 which are for example used in various processing carried out therein as described herein. A memory or computer-readable storage medium used in various embodiments may be a volatile memory, for example a DRAM (Dynamic Random Access Memory) or a non-volatile memory, for example a PROM (Programmable Read Only Memory), an EPROM (Erasable PROM), EEPROM (Electrically Erasable PROM), or a flash memory, e.g., a floating gate memory, a charge trapping memory, an MRAM (Magnetoresistive Random Access Memory) or a PCRAM (Phase Change Random Access Memory).

In various embodiments, a “circuit” may be understood as any kind of a logic implementing entity, which may be special purpose circuitry or a processor executing software stored in a memory, firmware, or any combination thereof. Thus, in an embodiment, a “circuit” may be a hard-wired logic circuit or a programmable logic circuit such as a programmable processor, e.g., a microprocessor (e.g., a Complex Instruction Set Computer (CISC) processor or a Reduced Instruction Set Computer (RISC) processor). A “circuit” may also be a processor executing software, e.g., any kind of computer program, e.g., a computer program using a virtual machine code, e.g., Java. Any other kind of implementation of the respective functions may also be understood as a “circuit” in accordance with various embodiments. Similarly, a “module” may be a portion of a system according to various embodiments and may encompass a “circuit” as described above, or may be understood to be any kind of a logic-implementing entity.

The present disclosure also discloses various systems (e.g., each may also be embodied as a device or an apparatus), such as the system 300 for tracking one or more living objects, the system 400 for tracking one or more living objects, for performing various operations/functions of various methods described herein. Such systems may be specially constructed for the required purposes, or may comprise a general purpose computer or other device selectively activated or reconfigured by a computer program stored in the computer. The algorithms presented herein are not inherently related to any particular computer or other apparatus. Various general-purpose machines may be used with computer programs in accordance with the teachings herein. Alternatively, the construction of more specialized apparatus to perform various method steps may be appropriate.

In addition, the present disclosure also at least implicitly discloses a computer program or software/functional module, in that it would be apparent to the person skilled in the art that individual steps of various methods described herein may be put into effect by computer code. The computer program is not intended to be limited to any particular programming language and implementation thereof. It will be appreciated that a variety of programming languages and coding thereof may be used to implement the teachings of the disclosure contained herein. Moreover, the computer program is not intended to be limited to any particular control flow. There are many other variants of the computer program, which can use different control flows without departing from the scope of the disclosure. It will be appreciated by a person skilled in the art that various modules described herein (e.g., the data processing module of system 300, the input module 412, the data sampling module 414, the data processing module 416) may be software module(s) realized by computer program(s) or set(s) of instructions executable by a computer processor to perform the required functions, or may be hardware module(s) being functional hardware unit(s) designed to perform the required functions. It will also be appreciated that a combination of hardware and software modules may be implemented.

Furthermore, two or more of the steps of a computer program/module or method described herein may be performed in parallel rather than sequentially. Such a computer program may be stored on any computer readable medium. The computer readable medium may include storage devices such as magnetic or optical disks, memory chips, or other storage devices suitable for interfacing with a computer. The computer program when loaded and executed on such the computer effectively results in a system or an apparatus that implements various steps of methods described herein.

In various embodiments, there is provided a computer program product, embodied in one or more computer-readable storage mediums (non-transitory computer-readable storage medium(s)), comprising instructions (e.g., the data processing module of the system 300) executable by one or more computer processors to perform the method 100 for tracking one or more living objects, as described herein with reference to FIG. 1 according to various embodiments. Accordingly, various computer programs or modules described herein may be stored in a computer program product receivable by a system therein, such as the system 300 for tracking one or more living objects as shown in FIG. 3, for execution by at least one processor 304 of the system 300 to perform various functions.

Similarly, in various embodiments, there is provided a computer program product, embodied in one or more computer-readable storage mediums (non-transitory computer-readable storage medium(s)), comprising instructions (e.g., the input module 412, the data sampling module 414, the data processing module 416) executable by one or more computer processors to perform the method 200 for tracking one or more objects, as described herein with reference to FIG. 2 according to various embodiments. Accordingly, various computer programs or modules described herein may be stored in a computer program product receivable by a system therein, such as the system 400 for tracking one or more living objects as shown in FIG. 4, for execution by at least one processor 404 to perform various functions.

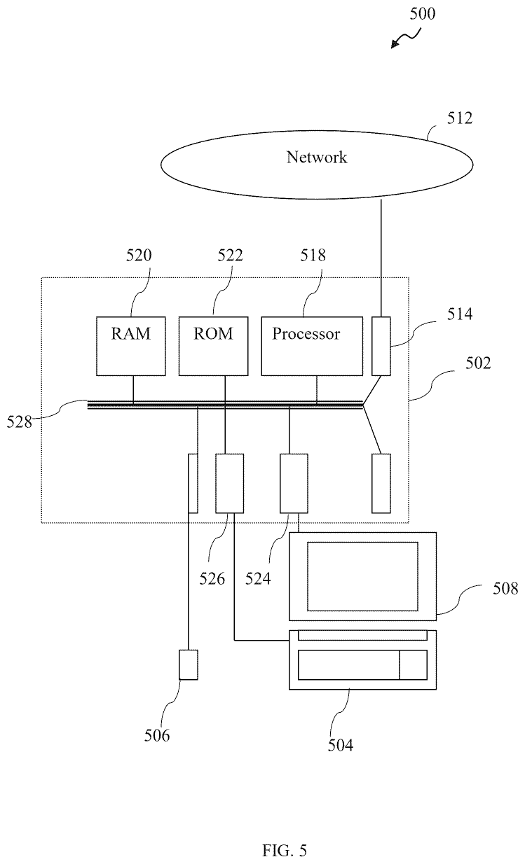

In various embodiments, the system 300, and/or the system 400 may each be realized by any computer system (e.g., desktop or portable computer system (e.g., mobile device)) including at least one processor and at least one memory, such as an example computer system 500 as schematically shown in FIG. 5 as an example only and without limitation. Various methods/steps or functional modules may be implemented as software, such as a computer program being executed within the computer system 500, and instructing the computer system 500 (in particular, one or more processors therein) to conduct various functions or operations as described herein according to various embodiments. The computer system 500 may comprise a system unit 502, input devices such as a keyboard and/or a touchscreen 504 and a mouse 506, and a plurality of output devices such as a display 508. The system unit 502 may be connected to a computer network 512 via a suitable transceiver device 514, to enable access to e.g., the Internet or other network systems such as Local Area Network (LAN) or Wide Area Network (WAN). The system unit 502 may include a processor 518 for executing various instructions, a Random Access Memory (RAM) 520 and a Read Only Memory (ROM) 522. The system unit 502 may further include a number of Input/Output (I/O) interfaces, for example I/O interface 524 to the display device 508 and 110 interface 526 to the keyboard 504. The components of the system unit 502 typically communicate via an interconnected bus 528 and in a manner known to a person skilled in the art.

In order that the present disclosure may be readily understood and put into practical effect, various example embodiments of the present disclosure will be described hereinafter by way of examples only and not limitations. It will be appreciated by a person skilled in the art that the present disclosure may, however, be embodied in various different forms or configurations and should not be construed as limited to the example embodiments set forth hereinafter. Rather, these example embodiments are provided so that this disclosure will be thorough and complete, and will fully convey the scope of the present disclosure to those skilled in the art.

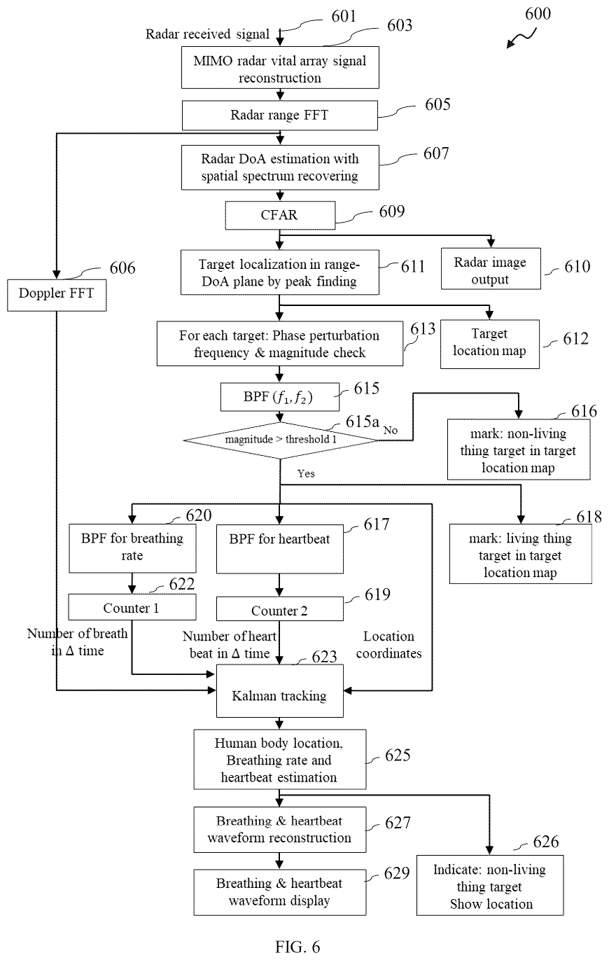

FIG. 6 depicts a schematic block diagram of a method 600 for tracking one or more living objects, according to various example embodiments of the present disclosure. The proposed VS detection and tracking method 600 as shown in FIG. 6 may include comprehensive radar detections. The proposed method 600 may be the joint breathing rate, heart beat and human position estimation and tracking. The method 600 may make use of the coherent information among these signals to increase the estimation reliability and accuracy. It may also overcome the overlapping pass-band issue of the fixed breathing rate and heart beat band pass filters (BPFs).

At step 601, signals transmitted from a plurality of transmitting antennas are received by a plurality of receiving antennas. The receiving antennas and transmitting antennas may include radar antennas.

At step 603, the received signals corresponding to multiple transmitting (Tₓ) and multiple receiving (Rₓ) antennas may be sampled to re-construct antenna array signal following multiple-input and multiple-output (MIMO) principle. That is, the received signals (rtf) may be sampled to generate a sequence of received signal values (V(n)) for each pair of transmit antenna (Tₓ) and receive antenna (Rₓ). For example, by using the basic re-construction algorithm, the reconstructed antenna array signal is

$$V ( n ) = Vec ( M ( n ) ) ∈ {ℂ}^{TR \times 1} , \qquad ( 1 )$$

where n denotes nth sample of the analog-to-digital conversion (ADC) output, Vec(·) means vectorization operation and

$$M ( n ) = [ \begin{matrix}{r}_{11} ( n ) & … & {r}_{T 1} ( n ) \\ ⋮ & {r}_{ij} ( n ) & ⋮ \\ {r}_{1 R} ( n ) & … & {R}_{TR} ( n )\end{matrix} ] , \qquad ( 2 )$$

and where rᵢⱼ represents the received signal corresponding to Tₓ antenna i's emission at Rₓ, antenna j. There are totally TTₓ antennas and R Rₓ antennas.

At step 605, frequency domain values are generated by applying a Fourier Transform (e.g. a Fast Fourier Transform (FFT)) to the sequence of received signal values. The range FFT length is set as M. From each channel of the antenna array (column vector) V, form a block taking M sequential samples of a chirp duration, and conduct FFT. The kth range FFT output is

$${R}^{( i )} ( k ) = {ℱ}_{row} ( {D}_{range}^{( i )} ( k ) ) ∈ {ℂ}^{1 \times M} , \qquad ( 3 )$$

where (·)⁽ⁱ⁾ denotes from the ith channel of the antenna array, v⁽ⁱ⁾ is signal from the channel i of V. k denotes kth block. [symbol] _{row}( ) is row-wise 1D-FFT operator and

$${D}_{range}^{( i )} ( k ) = [ {v}^{( i )} ( ( k - 1 ) * M + 1 ) , … , {v}^{( i )} ( ( k - 1 ) * M + M ) ] \qquad ( 4 )$$

is the kth range FFT input block.

At step 607, a radar imaging result is generated by applying a spatial spectrum recovering algorithm to the frequency domain values to generate a first radar imaging result. This may be conducted among antenna array channels. The spatial spectrum recovering algorithm may include steering vector beamforming, capon and the like. The capon method is described as an example for the spatial spectrum recovering as below.

A matrix H is formed as below:

$$H ( k , m ) = [ \begin{matrix}{r}^{( 1 )} ( k , m ) , … , {r}^{( 1 )} ( k + Z - 1 , m ) \\ … \\ {r}^{( i )} ( k , m ) , … , {r}^{( i )} ( k + Z - 1 , m ) \\ … \\ {r}^{( TR )} ( k , m ) , … , {r}^{( TR )} ( k + Z - 1 , m )\end{matrix} ] \qquad ( 5 )$$

where Z is the time window size. r⁽ⁱ⁾(k, m) is the mth element of the kth range FFT output R⁽ⁱ⁾(k). S is set as the steering vector as below (6). A steering vector may represent the set of phase delays an incoming wave experiences, evaluated at a set of array elements (antennas).

$$S ( θ ) = {[ 1 , {e}^{\frac{j 2 π {f}_{c} d}{c} sin ( θ )} , … , {e}^{\frac{j 2 π {f}_{c} d}{c} ( TR - 1 ) sin ( θ )} ]}^{T} \qquad ( 6 )$$

The kth radar image is as

$$image ( k ) = [ \begin{matrix}\frac{1}{{S}^{H} ( - \frac{π}{2} ) {C}^{- 1} ( k , m ) S ( - \frac{π}{2} )} , … , \frac{1}{{S}^{H} ( - \frac{π}{2} ) {C}^{- 1} ( k , M ) S ( - \frac{π}{2} )} \\ \frac{1}{{S}^{H} ( - \frac{π}{2} + ϵ ) {C}^{- 1} ( k , m ) S ( - \frac{π}{2} + ϵ )} , … , \frac{1}{{S}^{H} ( - \frac{π}{2} + ϵ ) {C}^{- 1} ( k , M ) S ( - \frac{π}{2} + ϵ )} \\ ⋮ \\ \frac{1}{{S}^{H} ( \frac{π}{2} ) {C}^{- 1} ( k , m ) S ( \frac{π}{2} )} , … , \frac{1}{{S}^{H} ( \frac{π}{2} ) {C}^{- 1} ( k , M ) S ( \frac{π}{2} )}\end{matrix} ] ∈ {ℂ}^{181 \times M} \qquad ( 7 )$$

where ϵ is the and e scanning step size, which is set as 1 degree, and

$$C ( k , m ) = H ( k , m ) {H}^{H} ( k , m ) \qquad ( 8 )$$

is the spatial covariance matrix calculated over Z sampling periods and (·)^{H} denotes the conjugated transpose. image(k) is the preliminary result of the radar imaging. The size of the radar image is

$$\frac{181}{ϵ} \times M = 181 \times M$$

It is updated every M sequential samples from all array channels.

Accordingly, a radar image may be formed based on the frequency domain values of the sequence of received signal values from the receiving radar antennas. The spatial spectrum recovering algorithm may be applied on the frequency domain values for radar imaging as a function of a set of phase delays of the received signal values (e.g. a steering vector), and a spatial covariance matrix calculated over Z sampling periods based on the frequency domain values and a conjugated transpose of the frequency domain values, to generate a preliminary result of the radar imaging.

At step 609, a constant false alarm rate (CFAR) algorithm may be applied to the preliminary radar imaging result generated by the spatial spectrum recovering algorithm. As described herein, the cell-average method as an example of the CFAR algorithm is presented below. The other CFARs algorithms may include the sophisticated ones using more accurate and non-Gaussian statistics models for the background noise in different application scenarios to determine the threshold.

In 181×M matrix, starting from top-left, scan is performed from left to right element-to-element and row by row. For an element, ζᵢⱼ in image(k), the threshold value c_{i,j} for ζᵢⱼ is calculated as

$${c}_{i , j} = mean ( abs ( [ \begin{matrix}{ζ}_{i - \frac{Δ}{2} , j - \frac{Δ}{2}} , … , {ζ}_{i - \frac{Δ}{2} , j + \frac{Δ}{2}} \\ ⋮ \\ {ζ}_{i + \frac{Δ}{2} , j - \frac{Δ}{2}} , … , {ζ}_{i + \frac{Δ}{2} , j + \frac{Δ}{2}}\end{matrix} ] ) ) \qquad ( 9 )$$

where Δ is the block size and is an event number, abs( ) means taking absolute value element-wise, mean( ) is averaging operation for all elements. When

$$i - \frac{Δ}{2} < 0 or / and j - \frac{Δ}{2} < 0 ,$$

the corresponding element is zero-padding one. Zero-padding may be performed to obtain faster speed and/or improved accuracy. Then the CFAR output image is Image_{CFAR}(k) with every element

$${ζ}_{i , j}^{′} = \{ \begin{matrix}abs ( {ζ}_{i , j} ) , & if abs ( {ζ}_{i , j} ) > {c}_{i , j} \\ 0 , & if abs ( {ζ}_{i , j} ) \leq {c}_{i , j}\end{matrix} \qquad ( 10 )$$

image_{CFAR}(k) is the output of the radar imaging with unwanted background noise removed. It is a 181×M matrix and updated every M sequential samples from all antenna array channels.

Accordingly, a further imaging function is applied to the preliminary result of the radar imaging. The further imaging function may include determining absolute values of elements in the preliminary result and determining an average of the absolute values as a threshold value. The absolute values may be determined within a predetermined block size with respect to a number of elements (e.g. not all the elements in the preliminary result). A larger block size may be preferred for better results (e.g. where a living object is identified). The further imaging function may further include determining an further element equal to the absolute value of the element if the absolute value of an element is greater than the threshold value and determining an further element equal to zero if the absolute value of an element is less than or equal to the threshold value. The CFAR output image may be improved by removing unwanted background noise which corresponds to the zero-value further elements.

At step 610, the radar image (i.e. CFAR output image) is output.

At step 611, 2-dimension (2D) peak finding is conducted in image_{CFAR}(k) matrix. The peak locations in image_{CFAR}(k) are the detection target locations. The detected peaks in image_{CFAR}(k) may be also peaks in Image(k). The detected number of targets is set as X. The detection X targets are denotes as, P₁, . . . Pₙ, . . . , Pₓ, and the location of Pₙ(k) in image_{CFAR}(k) is denoted as (xₙ(k), yₙ(k)), where, xₙ(k)=range(Pₙ(k))sin (DoA(Pₙ(k))), yₙ(k)=range(Pₙ(k))cos(DoA(Pₙ(k))). DoA represents direction-of-arrival estimation. Accordingly, the locations of the detected targets may be obtained by performing direction-of-arrival (DoA) estimation on the CFAR output image as shown in (10).

At step 612, a target location map is output showing the locations of the targets (e.g. mapped by peaking finding)

At step 613, a phase perturbation (i.e. v′ₙ(k)) of each detected peak for each discrete time k in multiple detection times (Z sampling periods) is determined to generate unwrapped phase angles. The phase perturbation at every detected target location in Image(k) along time index k is calculated as below:

$${v}_{n} ( k ) = ∠ ( {S}^{H} ( DoA ( {P}_{n} ( k ) ) ) {X}_{n} ( k ) ) \qquad ( 11 )$$

where ∠(·) denotes the phase angles of the complex number ‘·’, and Xₙ(k)=[r⁽¹⁾(k, range(Pₙ(k))), . . . , r^{(TR)} k,range (Pₙ(k)))]^{T} representing the location of the X target with respect to discrete time k. S^{H} is the conjugate transpose of the steering factor S in equation (6).

Conducting sliding window unwrapping with window size Z as below:

$${v}_{n}^{′} ( k ) = {V}_{z} , \qquad ( 12 )$$

where V_{z} is the zth element of vector V and

$$V = unwrap ( [ {v}_{n} ( k ) , … , {v}_{n} ( k + Z - 1 ) ] ) , \qquad ( 13 )$$

and where unwrap(·) means unwrapping the radian phase angles in a vector.

Accordingly, determining the phase perturbation of each detected peak may include determining DoA estimation on the location of each (P₁, . . . Pₙ, . . . , Pₓ) of the X targets. Determining the phase perturbation of each detected peak may further include determining phase angles as a function of the DoA estimation on the location of each (P₁, . . . Pₙ, . . . , Pₓ) of the X targets, the location of each (P₁, . . . Pₙ, . . . , Pₓ) of the X targets, and the conjugate transpose of the steering factor S^{H}, and unwrapping the phase angles (e.g. restoring the physical continuity of the phase map) with respect to frequency.

At step 615, band pass filtering (BPF) is performed on the generated unwrapped phase angles for each peak. For every v′ₓ(k), band pass filtering may be conducted with a first frequency f₁=0.1 Hz, and a second frequency f₂=4 Hz:

$${v}_{n}^{″} ( k ) = BPF ( {v}_{n}^{′} ( k ) ) \qquad ( 14 )$$

As step 615a, a peak is determined to correspond to a living object if a measure of the filtered unwrapped phase angles for the peak is above a predetermined threshold (γ). For every v″ₙ(k) for X targets (e.g. n=1 to X), if

$$‖ {V}_{n}^{″} ( k ) ‖ > γ , where \qquad ( 15 )$$

$${V}_{n}^{″} ( k ) = [ {v}_{n}^{″} ( k ) , … , {v}_{n}^{″} ( k + Z - 1 ) ] \qquad ( 16 )$$

and γ is a VS threshold. Then mark target n as a living object (step 618), otherwise, mark target n as a non-living object (step 616). In the localization map, plot all target locations (P₁, . . . Pₙ, . . . Pₓ), and mark their respective types (living objects or non-living objects).

Accordingly, the measure of the filtered unwrapped phase angles for the peak may include taking the norm of the quantity of the filtered unwrapped phase angles with respect to discrete time k during the Z sampling periods.

At step 606, Doppler domain values (e.g., moving target speeds) are determined for the frequency domain values:

$${D}^{( i )} ( k ) = {ℱ}_{row} ( [ {R}^{{( i )}^{T}} ( k ) , … , {R}^{{( i )}^{T}} ( k + Z - 1 ) ] ) ∈ {ℂ}^{M \times Z} \qquad ( 17 )$$

where [°]^{T} denotes transpose matrix ‘°’. Then the Doppler speeds for targets 1 to X are determined by their Doppler FFTs at their ranges, respectively. The Doppler FFT result may be used as \vec{u}ₙ(k) in equation (27).

At steps 617 and 620, for all living object targets' BPF signals, conduct a low pass filter (LPF) with a third frequency f_{L}=0.5 Hz and a high pass filtering (HPF) with a fourth frequency f_{H}=0.5 Hz in parallel. The outputs are

$${B}_{n , m} ( k ) = LPF ( {v}_{n}^{″} ( k ) ) , and \qquad ( 18 )$$

$${H}_{n , m} ( k ) = HPF ( {v}_{n}^{″} ( k ) ) , \qquad ( 19 )$$

where n is the index of the targets, m is the index of living object targets, and assuming there are X′ living thing targets detected.

Let

$${B}_{n , m}^{′} ( k ) = \{ \begin{matrix}{B}_{n , m} ( k ) , & if {B}_{n , m} ( k ) > 0.5 \overline{\vert {B}_{n , m} ( k ) \vert} \\ 0 , & if {B}_{n , m} ( k ) \leq 0.5 \overline{\vert {B}_{n , m} ( k ) \vert}\end{matrix} , and \qquad ( 20 )$$

$${H}_{n , m}^{′} ( k ) = \{ \begin{matrix}{H}_{n , m} ( k ) , & if {H}_{n , m} ( k ) > 0.5 \overline{\vert {H}_{n , m} ( k ) \vert} \\ 0 , & if {H}_{n , m} ( k ) \leq 0.5 \overline{\vert {H}_{n , m} ( k ) \vert}\end{matrix} , where \qquad ( 21 )$$

$$\overline{\vert {B}_{n , m} ( k ) \vert} = mean ( [ {B}_{n , m} ( k ) , … , {B}_{n , m} ( k + Z - 1 ) ] ) , and \qquad ( 22 )$$

$$\overline{\vert {H}_{n , m} ( k ) \vert} = mean ( [ {H}_{n , m} ( k ) , … , {H}_{n , m} ( k + Z - 1 ) ] ) . \qquad ( 23 )$$

mean(·) denotes averaging among all element of vector ‘·’. Then, the initial detections for the numbers of breaths and heartbeats during the Z sampling period are

$${C}_{{B}_{m}} ( k ) = size ( findpeaks ( [ {B}_{n , m}^{′} ( k ) , … , {B}_{n , m}^{′} ( k + Z - 1 ) ] ) ) , and \qquad ( 24 )$$

$${C}_{{H}_{m}} ( k ) = size ( findpeaks ( [ {H}_{n , m}^{′} ( k ) , … , {H}_{n , m}^{′} ( k + Z - 1 ) ] ) ) , \qquad ( 25 )$$

respectively, where findpeaks(·) denotes finding peaks in vector ‘·’, and size(·) denotes the number of elements in vector ‘·’.

Accordingly, a breathing rate data (B′_{n,m}(k)) may be modulated equal to the raw breathing rate (B_{n,m}(k)) if the raw breathing rate (B_{n,m}(k)) is greater than a predetermined ratio (e.g., 0.5) of an average ( [symbol] ) of the raw breathing rates (B_{n,m}(k)) during the sampling period (Z). A breathing rate (B′_{n,m}(k)) may be modulated to zero if the raw breathing rate (B_{n,m}(k)) is less than or equal to predetermined ratio (e.g., 0.5) of the average ( [symbol] ) of the raw breathing rates (B_{n,m}(k)) during the sampling period. A breathing rate (C_{B}_{ₘ}(k)) of a living object may be determined by finding peaks in the breathing rate data during the sampling period and sizing the number of the peaks of the breathing rate data.

Accordingly, a heartbeat data (H′_{n,m}(k)) may be modulated equal to the raw heartbeat (H_{n,m}(k)) if the raw heartbeat (H_{n,m}(k)) is greater than a predetermined ratio (e.g., 0.5) of an average ( [symbol] ) of the raw heartbeats (H_{n,m}(k)) during the sampling period (Z). A heartbeat (H_{n,m}(k)) may be modulated to zero if the raw heartbeat (H_{n,m}(k)) is less than or equal to predetermined ratio (e.g., 0.5) of the average ( [symbol] ) of the raw heartbeats (H_{n,m}(k)) during the sampling period. A heartbeat (C_{H}_{ₘ}(k)) of a living object may be determined by finding peaks in the heartbeat data during the sampling period and sizing the number of the peaks of the heartbeat data.

It should be appreciated that although the predetermined ratio is shown as 0.5 in the equations (20) and (21), other ratios may be determined in accordance with requirements, for example, 0.45, 0.4 or 0.6, or in a range of 0.45 to 0.55.

The determined breathing rates and heartbeats are output to counter 1 (step 622) and counter 2 (step 619), respectively.

At step 623, for every living object target n, conduct joint location, breathing rate and heartbeat tracking using Kalman filtering.

Let the state vector of the vehicle is

$${\vec{z}}_{n} ( k ) = [ \begin{matrix}{x}_{n} ( k ) \\ {v}_{{x}_{n}} ( k ) \\ {y}_{n} ( k ) \\ {v}_{{y}_{n}} ( k ) \\ {C}_{{B}_{n}} ( k ) \\ {v}_{{C}_{{B}_{n}}} ( k ) \\ {C}_{{H}_{n}} ( k ) \\ {v}_{{C}_{{H}_{n}}} ( k )\end{matrix} ] \qquad ( 26 )$$

where k denotes discrete time k, xₙ(k) and yₙ(k) denote the target n's position projected to X and Y axes in discrete time k, respectively. The time duration between any discrete times k and k−1 is β, which is Z times sampling duration, vₓ_{ₙ}(k) and v_{y}_{ₙ}(k) are the speeds of the target moving projected to X and Y axes, respectively. C_{B}_{ₙ}(k) and C_{H}_{ₙ}(k) are the breathing rate and heartbeat of living object target n initially detected by radar sensor with respect to discrete time k, and v_{C}_{_{Bn}}(k) and V_{C}_{_{Hn}}(k) are the changing rates of the breathing rate and heartbeat of living thing target n with respect to discrete time k. These elements (including xₙ(k) and yₙ(k), vₓ_{ₙ}(k) and v_{y}_{ₙ}(k), C_{B}_{ₙ}(k) and C_{H}_{ₙ}(k), v_{C}_{_{Bn}}(k) and v_{C}_{_{Hn}}(k)) in the state vector \vec{z}ₙ(k) may be updated in the below equation (27). Since the state vector includes heartbeat, breathing rate, their change rates as well as the object location moving speeds, the state vector may build the relationship between the moving speed and vital sign values, and achieve data fusion through tracking this state vector including the object speeds. In other words, by tracking the state vector that includes multiple inter-related datasets, the proposed method provides more accurate and consistent detection.

Accordingly, the state vector with respect to discrete time k+1 is represented as:

$${\vec{z}}_{n} ( k + 1 \vert k ) = F {\vec{z}}_{n} ( k \vert k ) + B {\vec{u}}_{n} ( k ) where \qquad ( 27 )$$

$$F = [ \begin{matrix}1 & β & 0 & 0 & 0 & 0 & 0 & 0 \\ 0 & 1 & 0 & 0 & 0 & 0 & 0 & 0 \\ 0 & 0 & 1 & β & 0 & 0 & 0 & 0 \\ 0 & 0 & 0 & 1 & 0 & 0 & 0 & 0 \\ 0 & 0 & 0 & 0 & 1 & β & 0 & 0 \\ 0 & 0 & 0 & 0 & 0 & 1 & 0 & 0 \\ 0 & 0 & 0 & 0 & 0 & 0 & 1 & β \\ 0 & 0 & 0 & 0 & 0 & 0 & 0 & 1\end{matrix} ] . \qquad ( 28 )$$

The control vector is

$${\vec{u}}_{n} ( k ) = [ \begin{matrix}0 \\ {D}_{n} sin ( DoA ( {P}_{n} ) ) \\ 0 \\ {D}_{n} cos ( DoA ( {P}_{n} ) ) \\ 0 \\ 0 \\ 0 \\ 0\end{matrix} ] , and \qquad ( 29 )$$

$$B = [ \begin{matrix}0000 … 0 \\ 0100 … 0 \\ 0000 … 0 \\ 0001 … 0 \\ 0000 … 0 \\ ⋮ \\ 0000 … 0\end{matrix} ] \qquad ( 30 )$$

where Dₙ is the Doppler speed of target Pₙ detected in equation (17) by radar, DoA(Pₙ) is the DoA of Pₙ detected by radar. And

$${\vec{m}}_{n} ( k ) = H {\vec{z}}_{n} ( k ) , \qquad ( 31 )$$

$${\vec{m}}_{n} ( k ) = [ \begin{matrix}{x}_{n} ( k ) \\ {y}_{n} ( k ) \\ {C}_{{B}_{n}} ( k ) \\ {C}_{{H}_{n}} ( k )\end{matrix} ] , \qquad ( 32 )$$

$$H = [ \begin{matrix}1 & 0 & 0 & 0 & 0 & 0 & 0 & 0 \\ 0 & 0 & 1 & 0 & 0 & 0 & 0 & 0 \\ 0 & 0 & 0 & 0 & 1 & 0 & 0 & 0 \\ 0 & 0 & 0 & 0 & 0 & 0 & 1 & 0\end{matrix} ] , \qquad ( 33 )$$

where \vec{m}(k) is the measure vector in discrete time k from radar.

Recursive procedure:
Predicted (a priori) state estimate: \vec{z}ₙ(k|k−1)=F\vec{z}ₙ(k−1|k−1)+B\vec{u}ₙ(k)
Predicted (a priori) estimate covariance: P(k|k−1)=FP(k−1|k−1)F^{T}+Q
measurement pre-fit residual: \tilde{y}(k)=\vec{m}ₙ(k)−H\vec{z}ₙ(k|k−1)
Innovation (or pre-fit residual) covariance: s(k)=HP(k|k−1)H^{T}+R
Optimal Kalman gain: Kal(k)=P(k|k−1)H^{T}S(k)¹
Updated (a posteriori) state estimate: \vec{z}ₙ(k|k)=\vec{z}ₙ(k|k−1)+Kal (k)\tilde{y}(k)
Updated (a posteriori) estimate covariance: P(k|k)=(1−Kal(k)H)P(k|k−1)

Then, the Kalman tracking outputs for target n are in \vec{Z}ₙ(k|k).

Accordingly, the method 600 may track the location (the target n's position projected to X and Y axes in discrete time k) of the one or more living objects (e.g. target 1, . . . , n, . . . , X), and the breathing rates and heartbeats (C_{B}_{ₙ}(k) and C_{H}_{ₙ}(k)) of one or more living objects with respect to the discrete time k. Further, the method 600 may further track the movement of one or more moving living objects by tracking the speeds (vₓ_{ₙ}(k) and v_{y}_{ₙ}(k)) of the target movement projected to X and Y axes. The method 600 may also track changing rates (v_{C}_{_{Bn}}(k) and V_{C}_{_{Hn}}(k)) of the breathing rates and heartbeats of living object target n with respect the discrete time k.

At step 625, the location and estimated VSs including breathing rates and heartbeats for the one or more living object targets are output, for example, to display the location in a target location map.

At step 626, one or more non-living object targets are indicated by showing location thereof.

At step 627, the waveforms of breathing rates and heartbeats for the one or more living object targets are reconstructed according to their estimated rates, respectively.

At step 629, the waveforms of breathing rates and heartbeats for the one or more living object targets are displayed.

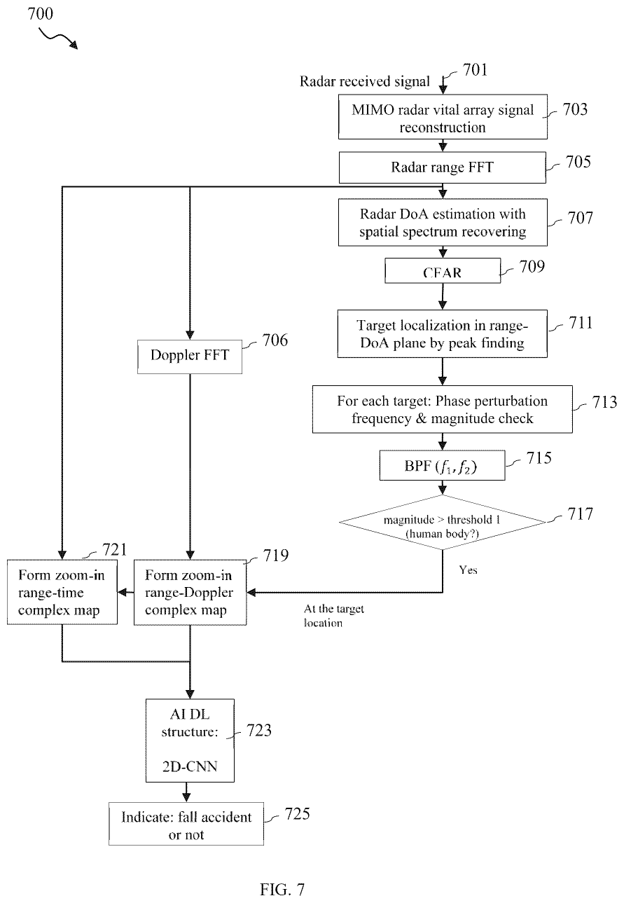

FIG. 7 depicts a schematic block diagram of a method 700 for tracking one or more living objects, according to various example embodiments of the present disclosure. The method 700 may include the similar steps as method 600, for example, steps 701, 703, 705, 706, 707, 709, 711, 713, 715 and 717 are similar to step 601, 603, 605, 606, 607, 609, 611, 613, 615 and 615a, and therefore, the similar steps need not be discussed. Features that are described in the context of the method 600 may correspondingly be applicable to the same or similar features in the method 700. Furthermore, additions and/or combinations and/or alternatives as described for a feature in the context of the method 600 may correspondingly be applicable to the same or similar feature in the method 700.

At step 719, zoom-in range-Doppler complex map is formed.

At step 721, zoom-in range-time complex map is formed.

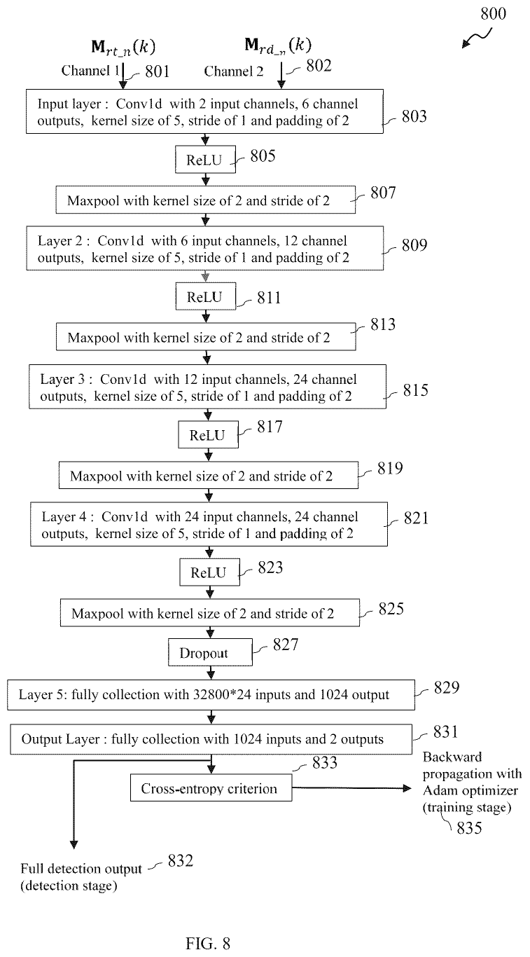

At step 723, the zoom-in range-Doppler complex map from step 719 and the zoom-in range-time complex map from step 721 will be analyzed by a deep learning structure as described with reference to FIG. 8.

Fall detection may be achieved through inference by the designed AI model instead of manually setting a criterion. Before the AI model to infer fall or not, the AI model may be trained by a real dataset including radar signals with falls or normal conditions. After the training, the AI model may directly infer whether there is fall with received new data.

At step 725, fall detection result is output.

The method 700 may be applied in a fall detection as a pre-condition that a human subject is detected first before a fall detection determination is activated. That is when the MIMO VS radar system detects the target as a human subject and localizes the human subject, then the fall detection may be activated at the specific location of the human subject. The method 700 may help to mitigate the need of wearables, sensors or video monitoring to detect falls, where such sensors are not suitable, for example, in a wet environment, or in shower where video monitoring raises privacy issues.

According to various non-limiting embodiments, if the target Pₙ is identified as a living object, at the location (DoA(Pₙ), range(Pₙ)) in DoA-range plane, the zoom-in complex maps Time-Range, Time-Doppler, Range-Doppler may be formed as follows.

The waveforms at the ADC sample k around location of target Pₙ in DoA-range plane is

$${\vec{w}}_{n} ( k ) = {S}^{H} ( DoA ( {P}_{n} ( k ) ) ) {X}_{n} ( k ) ∈ {ℂ}^{1 \times ( {Δ}_{r} + 1 )} where \qquad ( 34 )$$

$$S ( θ ) = {[ 1 , {e}^{\frac{j 2 π {f}_{c} d}{c} sin ( θ )} , … , {e}^{\frac{j 2 π {f}_{c} d}{c} ( TR - 1 ) sin ( θ )} ]}^{T} \qquad ( 35 )$$

is the steering vector (i.e. same as equation (6)), and

$${X}_{n} ( k ) = [ \begin{matrix}{r}^{( 1 )} ( k , range ( {P}_{n} ( k ) ) - \frac{{Δ}_{r}}{4} ) , … , {r}^{( 1 )} ( k , range ( {P}_{n} ( k ) ) + \frac{3 {Δ}_{r}}{4} ) \\ ⋮ \\ {r}^{( TR )} ( k , range ( {P}_{n} ( k ) ) - \frac{{Δ}_{r}}{4} ) , … , {r}^{( TR )} ( k , range ( {P}_{n} ( k ) ) + \frac{3 {Δ}_{r}}{4} )\end{matrix} ] \qquad ( 36 )$$

and r⁽ⁱ⁾(k, m) is the mth element of range FFT output R⁽ⁱ⁾(k) at sample k of antenna channel i as defined in equation (3) and Δᵣ, an even number, is the range window for the maps. Then the range-time map for target Pₙ is

$${M}_{rt \_ n} ( k ) = abs ( [ \begin{matrix}{\vec{w}}_{n} ( k ) \\ ⋮ \\ {\vec{w}}_{n} ( k + z )\end{matrix} ] ) ∈ {ℝ}^{z \times ( {Δ}_{r} + 1 )} , \qquad ( 37 )$$

where abs(·) denotes the absolute value of every element of the matrix ‘·’, and the range-Doppler map for target Pₙ is

$${M}_{{rd}_{\_ n}} ( k ) = abs ( {ℱ}_{column} ( {W}_{n} ( k ) ) ) ∈ {ℝ}^{z \times ( {Δ}_{r} + 1 )} , \qquad ( 38 )$$

where [symbol] _{column}(·) is column-wise 1D-FFT operator. In this disclosure, Z is selected as 12800 and Δᵣ as 40.

The fall detection may be formulated as a classification problem. There may be two classes: fall positive and fall negative. The deep learning structure may determinate which class is when the radar imaging map is given. As an example, the convolutional neural network (CNN) is used as the deep learning structure 800 as shown in FIG. 8. The parameter setting of the CNN is shown in FIG. 8. The input layer is a 1-dimension (1D) convolutional layer with 2 channel inputs (801, 802) and 6 channel outputs. The filter kernel size is 5, stride is 1 and padding is 2 (step 803). This is followed by ReLU activation (step 805) and a max pooling with kernel size of 2 and stride of 2 (step 807). Layer 2 is also a 1D convolutional layer with 6 channel inputs and 12 channel outputs. The filter kernel size is 5, stride is 1 and padding is 2 (step 809). This is followed by ReLU activation (step 811) and a max pooling with kernel size of 2 and stride of 2 (step 813). Layer 3 is also a 1D convolutional layer with 12 channel inputs and 24 channel outputs. The filter kernel size is 5, stride is 1 and padding is 2 (step 815). This is followed by ReLU activation (step 817) and a max pooling with kernel size of 2 and stride of 2 (step 819). Layer 4 is still a 1D convolutional layer with 24 channel inputs and 24 channel outputs. The filter kernel size is 5, stride is 1 and padding is 2 (step 821). This is followed by ReLU activation (step 823) and a max pooling with kernel size of 2 and stride of 2 (step 825).

After 4 convolutional layers, at step 827, a dropout operation is performed to enhance the reliability and to avoid the overfitting during the training.

After the dropout operation, at step 829, the layer 5 is a fully connected layer with 32800 times 24 inputs and 1024 outputs. Note that the sizes of range-time and range-Doppler map are all 41 times 12800. After max poolings in the 4 convolutional layers, the size of each channel of the latest convolutional layer is

$$41 \times \frac{12800}{{2}^{4}} = 32800.$$

The final output layer is also a fully-connected layer with 1024 inputs and 2 outputs (step 831). The full detection is output at step 832.

The deep learning structure may need to be trained before real detection. Because this is a classification problem, the criterion of the loss function may be cross-entropy (step 833). The optimizer for backward propagation may be Adam (step 835).

The method 700 based on the above embodiments may advantageously identify whether the detected target is a human body according to the vital sign first, followed by localizing the human body, and then conducting the fall accident detection at the human body location. Thus, it may eliminate the false alarm caused by non-living movement interference. Furthermore, it may localize the accident point and make multiple fall accident detection possible.

While the methods 600, 700 described above is illustrated and described as a series of steps or events, it will be appreciated that any ordering of such steps or events are not to be interpreted in a limiting sense. For example, some steps may occur in different orders and/or concurrently with other steps or events apart from those illustrated and/or described herein. In addition, not all illustrated steps may be required to implement one or more aspects or embodiments described herein. Also, one or more of the steps depicted herein may be carried out in one or more separate acts and/or phases.

While embodiments of the disclosure have been particularly shown and described with reference to specific embodiments, it should be understood by those skilled in the art that various changes in form and detail may be made therein without departing from the scope of the disclosure as defined by the appended claims. The scope of the disclosure is thus indicated by the appended claims and all changes which come within the meaning and range of equivalency of the claims are therefore intended to be embraced.

1. A method for tracking one or more living objects using at least one processor, the method comprising:
receiving, via a plurality of receiving antennas, signals transmitted from a plurality of transmitting antennas;
identifying a location of each of the one or more living objects with respect to a discrete time based on the signals received in a number of sampling periods;
determining a movement of each of the one or more living objects based on the identified location;
determining one or more vital signs of each of the one or more living objects based on the movement of each of the one or more living objects; and
tracking collectively the location and the one or more vital signs of each of the one or more living objects.

2. The method of claim 1, wherein the one or more vital signs comprises a breathing rate and/or a heartbeat of the one or more living objects.

3. The method of claim 2, wherein the one or more vital signs further comprises a changing rate of the breathing rate and/or a changing rate of the heartbeat of the one or more living objects.

4. The method of claim 1, wherein the movement of each of the one or more living objects comprises information relating a speed of each of the one or more living objects.

5. The method of claim 4, wherein the information relating a speed of each of the one or more living objects comprises a Doppler speed of each of the one or more living objects and Direction of Arrival (DOA) estimation of each of the one or more living objects.

6. The method of claim 2, wherein determining one or more vital signs of each of the one or more living objects comprises:
conducting a low pass filtering to obtain information relating to the breathing rate of the one or more living objects and/or conducting a high pass filtering to obtain information relating the heartbeat of the one or more living objects; and
determining the breathing rate of the one or more living object by finding peaks in the information relating to the breathing rate of the one or more living objects during the sample period and/or determining the heartbeat of the one or more living objects by finding peaks in the information relating to the heartbeat of the one or more living objects during the sample period.

7. The method of claim 6, wherein the low pass filtering and the high pass filtering are conducted at a same frequency.

8. The method of claim 6, further comprising:
determining an intermediate parameter equal to the low pass filtered information relating to the breathing rate of the one or more living objects if the low pass filtered information is greater than a predetermined ratio of an average of the low pass filtered information during the sample period and determining the intermediate parameter to zero if the low pass filtered information is less than or equal to predetermined ratio of the average of the low pass filtered information; and/or
determining an intermediate parameter equal to the high pass filtered information relating to the heartbeat of the one or more living objects if the high pass filtered information is greater than a predetermined ratio of an average of the high pass filtered information during the sample period and determining the intermediate parameter to zero if the high pass filtered information is less than or equal to predetermined ratio of the average of the high pass filtered information.

9. The method of claim 1, further comprising:
detecting a fall by using the convolutional neural network (CNN).

10. The method of claim 1, wherein identifying a location of each of the one or more living objects with respect to a discrete time based on the signals received in a number of sampling periods comprises:
sampling the received signals to generate a sequence of received signal values for each pair of transmitting antenna and receiving antenna;
generating frequency domain values by applying a Fourier Transform to the sequence of received signal values;
generating a radar imaging result by applying a spatial spectrum recovering algorithm to the frequency domain values to generate a first radar imaging result;
detecting peaks in the radar imaging result;
determining a phase perturbation of each detected peak to generate unwrapped phase angles;
performing band pass filtering of the generated unwrapped phase angles for each peak; and
determining a peak to correspond to a living object if a measure of the filtered unwrapped phase angles for the peak is above a predetermined threshold.

11. The method of claim 1, further comprising:
displaying the location of each of the one or more living objects in a target location map; and
indicating a location for each of one or more non-living object in the target location map.

12. The method of claim 1, further comprising:
displaying waveforms of the vital signs of the one or more living objects in accordance with estimated rates thereof.

13. The method of claim 1, further comprising, for every living object of the one or more living objects:
simultaneously identifying the location with respect to the discrete time based on the signals received in a number of sampling periods;
simultaneously determining the movement based on the identified location;
simultaneously determining the one or more vital signs based on the movement; and
simultaneously tracking collectively the location and the one or more vital signs using Kalman filtering.

14. A method for tracking one or more living objects using at least one processor, the method comprising:
receiving, via a plurality of receiving antennas, signals transmitted from a plurality of transmitting antennas;
sampling the received signals to generate a sequence of received signal values for each pair of transmitting antenna and receiving antenna;
generating frequency domain values by applying a Fourier Transform to the sequence of received signal values;
detecting peaks in the frequency domain values with respect to a discrete time based on signals received in a number of sampling periods;
determining a phase perturbation of each detected peak to generate unwrapped phase angles;
performing band pass filtering of the generated unwrapped phase angles for each peak; and
determining a peak to correspond to a living object if a measure of the filtered unwrapped phase angles for the peak is above a predetermined threshold.

15. The method of claim 14, prior to detecting peaks in the frequency domain values, further comprising:
generating a radar imaging result by applying a spatial spectrum recovering algorithm to the frequency domain values to generate a first radar imaging result;
wherein the spatial spectrum recovering algorithm gives a preliminary radar imaging result and generating the radar imaging result further comprises applying a constant false alarm rate (CFAR) algorithm to the preliminary radar imaging result.

16. The method of claim 14, further comprising:
determining Doppler domain values of the frequency domain values.

17. The method of claim 14, further comprising:
conducting a low pass filtering with a third frequency on the filtered unwrapped phase angles to generate raw breath rates;
modulating a breath rate data equal to the raw breath rate if the raw breath rate is greater than a predetermined ratio of an average of the raw breath rates during a sampling period and determining a breath rate data to zero if the raw breath rate is less than or equal to predetermined ratio of the average of the raw breath rates during the sampling period; and
determining a breath rate of a living object by finding peaks in the breath rate data during the sampling period and sizing the number of the peaks of the breath rate data.

18. The method of claim 14, further comprising:
conducting a high pass filtering with a fourth frequency on the filtered unwrapped phase angles to generate raw heartbeats;
modulating a heartbeat data equal to the raw heartbeat if the raw heartbeat is greater than a predetermined ratio of an average of the raw heartbeats during a sampling period and modulating a heartbeat data to zero if the raw heartbeat is less than or equal to predetermined ratio of the average of the raw heartbeats during the sampling period; and
determining a heartbeat of a living object by finding peaks in the heartbeat data during the sampling period and sizing the number of the peaks of the heartbeat data.

19. The method of claim 14, further comprising:
forming zoom-in range-time map and zoom-in range-Doppler map; and
processing the maps by deep learning structure.

20. A system for tracking living objects, the system comprising:
at least one memory; and
at least one processor communicatively coupled to the at least one memory and configured to perform the method according to claim 1.

21. (canceled)
